(19) United States
(12) Patent Application Publication
(10) Pub. No.: US 2009/0162920 A1
(43) Pub. Date: Jun. 25, 2009

(54) MODULAR CONTINUOUS PRODUCTION OF MICRO-ORGANISMS

(75) Inventors: Jan Vanhoutte, Zwevezele (BE); Koenraad Vanhoutte, Lochristi (BE)

Correspondence Address: DARBY & DARBY P.C., P.O. BOX 770, Church Street Station, New York, NY, 10008-0770, US

(73) Assignee: SBAE INDUSTRIES NV, Waarschoot (BE)

(21) Appl. No.: 12/210,835

(22) Filed: Sep. 15, 2008

Related U.S. Application Data
(63) Continuation of application No. PCT/EP2007/002290, filed on Mar. 15, 2007.

(30) Foreign Application Priority Data
Mar. 15, 2006 (EP) 06111173.8

(51) Int. Cl. C12N 1/20; C12M 1/00

(52) U.S. Cl. 435/261; 435/243; 435/308.1

(57) ABSTRACT

Process for the growing of micro-organism in an open and continuous system further being characterized by means for creating within said system a habitat in which natural, diverse and heterogeneous emicrobial communities can autonomously react and adapt to a changing environment

### CROSS REFERENCE TO PRIOR APPLICATIONS

This application is a PCT Continuation of International Application No. PCT/EP2007/002290, filed Mar. 15, 2007, which claims priority from European Application No. 06111173.8, filed Mar. 15, 2006. The aforementioned applications are incorporated herein by reference in their entireties. The International Application was published in English on Sep. 20, 2007 as WO 2007/104564 under PCT Article 21(2).

### BACKGROUND

For almost half a century extensive studies in both Europe and the US have been performed with regard to the feasibility to grow large quantities of micro-organisms (also referred to as ‘microbial organisms’) (i.e. metric tons). Two important questions in the research into growing such large volumes of micro-organisms (e.g. algae) were firstly, whether it was possible to use algae as a biological component in the treatment procedures of waste-streams and secondly, whether it was sensible to use cultured micro-organisms as a feedstock to generate new products, such as renewable bio-fuels. It has been conclusively demonstrated, both theoretically and practically, that growing micro-organisms, such as algae, has the potential to both efficiently treat waste-streams (e.g. waste-water) at large scales and produce sufficiently large quantities of feedstock for new products, such as renewable bio-fuels.

The major challenge remains to find an efficient and economical process for the production of microorganism, in particular algae and biomass in general. Primary requisite to this challenge is the optimized inoculation and production including the harvesting of these micro-organisms.

The present invention meets the above challenge by using the so-called complex adaptive system (CAS) approach and translating the CAS principle into a practical workable engineered open and continuous system thereby creating a simulation of a natural environment (habitat) for growing micro-organisms. In accordance with the process of the present invention the natural microbial communities are allowed to dynamically adapt to a changing simulated environment. Within the process of the present invention the communities composition changes autonomously and accordingly communities adapt themselves to the changing simulating environmental conditions. The autonomous adaptation and local interaction results in self-organization of the communities. As a result, an optimal habitat is created by the present invention to which natural and diverse microbial communities can autonomously react and adapt resulting in an optimized production of biomass. In contrast with the classic production of mono-culturing techniques, the present invention using the natural and divers communities allows for a more stable habitat, in turn resulting in an improved biomass production. In addition, the process of the present invention allows different communities to be created at different positions within the process resulting in a modular continuous production of microorganism.

In accordance with a first embodiment of the process of the present invention, an optimal habitat is simulated to which natural microbial communities can autonomously react and adapt. Those micro-organisms best suited to the environmental conditions in the growth recipient will thrive. In contrast with mono-culturing techniques, the process of the present invention employs diverse natural communities as a result of which the production of micro-organisms becomes more stable (e.g. in terms of biomass production).

In contrast with conventional algal culturing techniques whereby selected micro-organism are grown as a monoculture in a closed facility, in which almost all variables are very narrowly and rigidly fixed (often in function of productivity), the present invention creates a simulation of a natural environment (habitat) and then allows the organisms themselves to choose where and how to grow and thus adapt as communities to this simulated natural environment. In accordance with the present invention, micro-organisms which at a given time fail to colonize a certain position within the system are carried further downstream where they may encounter a suitable colonization-site or may eventually even be discarded as being superfluous. If at a later point in time the environmental conditions at the former given position have changed these micro-organisms may again colonize that spatial position. Accordingly, the present invention and its process configuration including system configuration allows for different communities to exist simultaneously at various positions within the total process of the present invention.

In accordance with another embodiment of the process of the present invention is directed to an open and continuous system whereas at present micro-algal culturing processes hitherto consist of closed circulatory batch systems (e.g. photo-bioreactors, high rate algal ponds). In this respect the invention is to be understood within the meaning of continuous whereas the prior technologies are limited to a ‘end point’. The water-stream treatment system of the present invention is open to the surrounding environment through its continuously maintained water stream. Furthermore, in accordance with the transversal nature of the process of the present invention, the interface between micro-organisms and nutrients in the water is continuously renewed while at the same time avoiding the necessity of reseeding the system after harvesting. In addition, as opposed to classic longitudinal systems, there will be a continuous presence of inocula in the flow path of the water including the entry point of the flow path.

In accordance with a further embodiment of the present invention, the process of the present invention employs artificial substrata positioned in the flow of the waste-stream forcing the stream through or near the artificial substrata and the microbial communities attached to them which increases the surface of interaction between growth medium (waste) and the microbial communities.

As a result of the process of the present invention, local microbial communities may be employed which facilitates up scaling and worldwide application. In contrast with present algal culturing techniques, the present invention allows a changing simulated environment (e.g. seasonality, nutrient-composition) whereby the natural microbial communities are allowed to dynamically adapt to the changing environment.

In conventional algal culturing techniques the micro-organism are introduced into the growth medium, whereas, in the present invention the growth medium is brought to and passed over and across the microbial communities and the nutrient-rich water or waste-stream is continuously renewed at the position where the micro-organisms grow.

In addition, it has been found that the harvesting procedure is greatly facilitated by introducing artificial substrata onto which the attached micro-organisms grow. By extracting the detachable carriers on which the artificial substrata are set the micro-organisms can be easily and efficiently separated from their growth medium (i.e. water such as waste-stream).

In line with the present invention, it has further surprisingly been found that the inoculation of artificial substrata positively influences the dynamics within the simulated habitat. These artificial substrata of the present invention enhance the processes of CAS and allow for interaction between the regional species pool and the communities within the system as well as enhance the mutually interactive process between the communities inside the system.

Production of microorganism, in particular, algae have been extensively described in the prior art with focus on waste water treatment. Prior art methods for growing biomass with a modular continuous method simulating the natural habitat of microorganism have not been recognized in the art nor have the specifics of the process of the present invention and its resulting benefits been disclosed nor suggested by the prior art.

### SUMMARY OF THE INVENTION

The present invention is directed to the growth of micro organism and to the improved production of biomass whereby attached microbial communities are grown in a continuous water stream in a controlled and directed fashion.

In accordance with another embodiment of the present invention, the process of the present invention employs artificial substrata preferably positioned transversal in the flow of the water stream forcing the stream through or near the artificial substrata and the microbial communities attached to them which increases the surface of interaction between growth medium (waste) and the microbial communities resulting in an optimize habitat for the communities to grow.

### DETAILED DESCRIPTION OF THE INVENTION

The present invention is directed to the production of the biomass whereby attached microbial communities are grown in a continuous water stream in a controlled and directed fashion.

### Use of Biological Processes and Natural Communities

According to the process of the present invention microbial communities that naturally occur in water including a waste-stream are grown. In case the water-stream is waste-water, this means that all organisms which occur in aquatic systems (natural or man-made) can in principle be observed in the biomass that is being cultured. The microbial communities that are cultured in this invention are natural, diverse and heterogeneous communities with all the natural dynamics of a wild microbial community. In this respect this invention differs from those technologies where carefully selected strains of organisms are cultured, either in mono-culture or poly-culture.

In accordance of the present invention, a simulated complex adaptive (CAS) system is created in a modular continuous manner. For the purpose of this invention it is understood that a community acting as a CAS has the abilities to autonomously adapt to changing environmental conditions. These environmental changes include, but are not limited to, changes in temperature, stream velocity, concentrations of various compounds, light intensity and periodicity, seasonal changes, . . . Adaptation of communities is a seemingly directed response by a community to any environmental change. This response may result in, but is not limited to, alteration of the community structure in terms of dominance, diversity, productivity, overall chemical composition, . . . The response of the community is the result of the compounded demographic changes (e.g. death, birth, immigration, emigration, . . . ) of all individual species and/or individual organisms that make up that community. As such, it can be said that communities adapt ‘as best as they can’ to the new environmental conditions without there being any human interference required. One possible measure of successful adaptation that could be considered is the viability of the individual cells of the microbial organisms, but many other measures can be devised for different purposes.

In accordance with the present invention, the applicant has successfully demonstrated and simulated CAS in a modulated continuous manner for the production of micro organism. In particular, two important aspects, where the universal biological principle of CAS is effectively simulated in accordance with the present invention, in illustrated in more detail:

Firstly, as the waste-stream passes through the artificial substrata harboring the microbial communities, the chemical composition of the waste-stream is altered by these microbial communities. This happens because the microbial communities deplete certain compounds in the water which are therefore of a lower concentration further downstream. Consequently, the environmental conditions are different at the consecutive spatial positions along the trajectory of the waste-stream. As microbial communities are CAS's, the microbial communities that grow at each spatial position will be those that are best adapted to the specific environmental conditions of that particular spatial position. The locally adapted microbial community has become dynamically self-organized. Therefore, microbial communities may differ at various spatial positions in various aspects e.g. diversity, dominance, . . . The distance between two spatial positions at which one microbial community differs from the next is not fixed but variable. It can in fact be different for each pair of spatial positions. The difference between microbial communities between spatial positions does not necessarily involve the same configuration of variables and/or parameters. For instance, between communities A and B the difference could be ‘diversity’, while between communities B and C it could be ‘productivity’ reflecting the modular continuous production of micro organism in accordance of the present invention.

The second important aspect where the biological principle of CAS is effectively simulated in accordance with the present invention entails the continuous selection of the microbial organisms entering and colonizing the artificial substrata of the present invention. As indicated already hereinabove, all microbial organisms that naturally occur within a region can be observed in the cultured biomass. All organisms that occur in natural microbial communities have some form of dispersal through which they can reach new habitats that are suitable for colonization. If upon arrival a habitat appears suitable it will be successfully colonized by that organism. Therefore, any habitat, comparable to some extent to a theoretical optimal habitat, will be continuously bombarded by ‘colonization particles’ further referred to as inocula. These inocula could take the form of seeds, spores, cysts, clumps of cells, . . . and can arrive actively on their own strength (e.g. flying insects) or passively by means of a vector (e.g. air, wind, water, attached to animals, . . . ).

The continuous new arrival of potential colonizers with the waste-stream allows microbial communities to regularly ‘recruit’ new organisms and incorporate them into the microbial community in order to be better adapted to the given environmental conditions. Local interactions between community components are essential in the self-organization of the adapting community. As these environmental conditions change through time additional organisms (species or groups) can be recruited into the community while others are lost from the community. Consequently, the microbial communities grown on the artificial substrata are continuously in contact with the regional communities through various complex processes bourn by the waste-stream. Some examples of such processes (but not limited to) are dispersal, colonization, recruitment and extinction.

The adaptation of the microbial communities occurs within the process of the present invention and the process is continuous and is not a temporally or spatially separated procedure or process. The microbial communities grown within the process of the present invention are recruited from regionally occurring microbial communities.

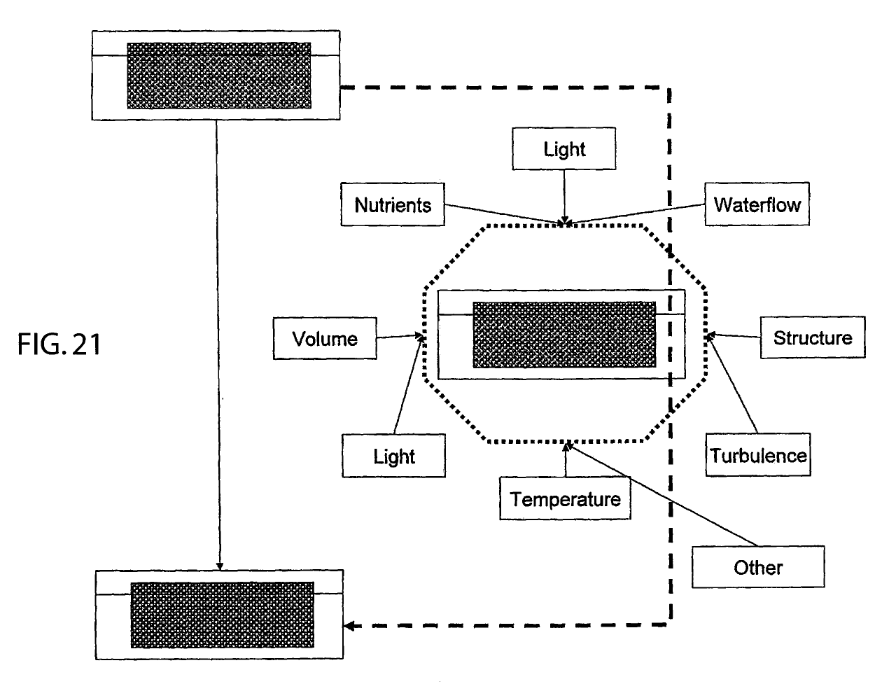

Over and above the simulation by the artificial substrata of the natural habitat (e.g. shape) by the present invention, the natural adaptation of the microbial communities to these artificial substrata and the general environmental conditions of the water (e.g. nutrients) and the region (e.g. climate), a number of additional environmental variables are controlled in this invention. These additional variables are carefully controlled and adjusted for the purpose of actively governing various properties of the microbial communities (e.g. physical, chemical, productivity, composition . . . ). For instance, by adjusting light the composition and dominance of the algal groups can be altered. Typical variables are, but not limited to, temperature, light spectrum, stream velocity and volume, nutrient and trace-element concentration, dissolved gasses: oxygen, carbon-dioxide, etc. (see FIG. 21).

The specific settings, governing and on-line controlling of these additional variables is to be regarded as a set of extra dimensions added to the habitat, to which the microbial communities will adjust autonomously, again through the principle of CAS. As such we refer to this approach as ‘modulation’ or ‘fine-tuning’ of the microbial communities that grow on the artificial substrata in the waste-stream resulting in a modular continuous production of micro organism.

### Microbial Communities

In accordance with the present invention a biomass is grown or cultured which consists predominantly of micro-organisms. For the purpose of this invention it is understood that micro-organisms are all organisms, both single-celled and multi-cellular organisms, of which the largest dimension is smaller than 2 mm. However, natural microbial communities typically harbor many organisms of which the size dimensions clearly exceed the 2 mm criteria, e.g. filamentous algae, nematodes. Therefore and for the purpose of this invention we explicitly state that the term ‘microbial communities’ is understood to include all larger organisms that naturally occur in or around these communities. This includes but is not limited to for instance filamentous algae, nematode worms, crustaceans, insects etc.

The biomass grown in accordance with the present invention comprises microbial communities in general and of certain groups of micro-organisms in particular. The determination of a ‘group’ can be based on a taxonomical, ecological or any other functional classification.

One possible preferred example of a biomass produced by this invention is a biomass consisting of attached microbial communities and dominated by the algal group Bacillariophyta or ‘diatoms’.

### Water-Stream

A water stream such as a waste-stream is any collection of chemical compounds present in a continuous stream. The stream can be in either a liquid and gaseous phase. The compounds can be used as essential growing nutrients by the microbial communities or can be secondarily immobilized by them, physical of chemical, either intra-cellular or in the matrix of microbial communities. Therefore, it is to be understood that for example a waste-stream is a liquid or gaseous stream of growth medium for microbial communities. At any given spatial position in the invention this stream of growth medium continuously replenishes the nutrients required for growth or the compounds to be immobilized.

The growth of the biomass and its presence in the waste-stream has a beneficiary effect on the waste-stream because it removes certain compounds from this stream and as such purifies the waste-stream of these compounds. These compounds may or may not be hazardous such as nitrates or toxic metals.

### Artificial Substrata

In accordance with the present invention, the growth of the biomass is directed in such a way that only attached microbial communities grow within the process of the present invention. These microbial communities are attached to and grown in and on artificial substrata, preferably volumetric spaced artificial substrata which simulate the natural substratum to which these communities naturally attach (e.g. sand-grains, plants, rocks, . . . ). Ideally, artificial substrata have a maximal attachment surface for a given volume by being fractal or fractal-like in shape and form without becoming so dissimilar from the natural substratum that the microbial communities no longer attach to them.

In accordance with the present invention, the substratum is placed within the stream, in such a way that the water flows over, across and through the substrata allowing for a transversal configuration of the system. The key problem of traditionally used substrata is that as biomass accrues on the substrata the system gets clogged and the flow is stopped, even up to a stage where the water passage is completely blocked. The flow in the process of the present invention is maintained because the fractal like nature of our substrata allows for the biomass to accrue on parts of the substrata whereas there are also wider openings that allow the flow to continue towards the next parts of the substrata. An example of execution is a carrier which holds a series of screens which form the substratum. These screens are perforated in a fractal patterns (the so called Serpienski Gasket). This patterns divides a triangle in 4 equal triangle. The central triangle is open, while the 3 outer are again perforated in the Serpienski pattern. This is repeated infinitesimally in theory. The biomass will start to settle in the smallest perforated zones which will clog up gradually, later the larger perforated zones will clog, but there will always be a central opening which will allow the water flow to continue. At a given point the surface will be clogged to a certain degree and there will be a drag on the screen caused by the flow. This drag can be measured by a device and can be used as an indicator for the ideal moment of harvesting. The carrier with substrata is then extracted by the extractor. This configuration of internal fractal patterning allows transversal positioning of the carriers with substrata. Fractal patterning other then on a triangular surface can equally be selected eg. square, hexagon etc. As in well understood in the art, the fractal patterns and forms can be exhibited on the outside (external) of a surface (eg. Koch Curve). Another configuration would be that the fractal patterning is distributed not on one single screen (or in one plane) but is continued over a number of sequential screens. This would in principle be a ‘3 D fractaloid’ or volumetric, whereas the above patterning on one screen is considered ‘2 D fractaloid’ or planar. From the figures the detailed fractal nature of the substrata and the transversal positioning which forces the water to flow through and across is further illustrated by a non-limiting example.

In accordance with the present invention, these artificial substrata are positioned in a waste-stream in such a manner that the waste-stream must pass through or close by the artificial substrata and consequently through or close by the microbial communities attached to these. The flow of the waste-stream is carefully controlled and governed.

The method of the present invention also comprises means for harvesting the microbial communities. Preferred harvesting the microbial communities comprise first of extracting the artificial substrata from the waste-stream by means of an extracting device. The present invention is also directed to such an extraction device to be used to extract the carriers holding the substrata said substrata attached to carriers, which aid in the fractal configuration of the system. This may be done either by taking hold of the artificial substratum directly or by taking hold of a carrier on which the artificial substrata were mounted before insertion into the waste-stream. Alternatively, the artificial substrata may be mounted on the carriers while the latter are already inserted into the waste-stream. The extracting device brings the artificial substrata to a harvesting device.

The attached microbial communities are subsequently separated from the artificial substrata in a harvesting device. In this harvesting device the separated microbial communities are concentrated using gravitation as primary principle. It is possible but not necessary to add an extra treatment phase between extraction of the artificial substrata and the separation of the biomass from the artificial substrata. This treatment (e.g. immersion in a treatment-fluid) is intended to increase the concentration of certain chemical compounds in the biomass (e.g. oil concentration). This is referred to as a two-step harvesting procedure. It should be understood that scraping processes used in biomass production could result in over harvesting along longitudinal sectors. In contrast, the present invention first extracts carriers with substrata, which results in transversal removing biomass, and then removing biomass from substrata can in fact use scraping or suchlike. In accordance with the present invention harvesting is monitored by judicious removal of particular sets of carriers with substrata from the stream whereby inadvertent over-harvesting is reduced.

### EXAMPLE

An example of an embodiment of the present invention is as follows:

Attached micro-algae are grown on brushes placed in a continuous stream of domestic wastewater derived from a traditional waste-water treatment facility. As the waste-water passes through the brushes, the growing algae actively decrease the concentration of e.g. nitrates and phosphates in the waste-water. Harvesting the algae is done in a washing machine in which the algae are sprayed from the brushes by water-jets. After separation from the brushes the algae will settle (i.e. gravity) on the bottom of the washing machine and can be taken from there for further processing. The intermediate step of a two-step harvesting procedure could for example entail immersing the brushes in a nutrient poor growth medium.

### Process Description of One Possible Embodiment

### The Treatment of a Waste-Water Stream by Growing Microbial Communities

Effluent water is derived from any water purification system consisting of a primary or secondary treatment phase. The effluent water could be derived from domestic, industrial and agricultural waste-water. In an ideal situation this effluent water is fully in compliance with the required environmental standards in the region of operation, as it is normally discharged into the waterways. At worst this invention may even operate with untreated water coming directly from the pollution source.

The waste-water is subsequently treated in the process of the present invention. If the process of the present invention is positioned after traditional waste-water treatment facilities, which would normally discharge the treated water, the treatment in the invention should be referred to as ‘a ternary water treatment phase’. For the purpose of ternary treatment the waste-water is pumped through a system of circulation, consisting of one or more recipients. The recipients are hereafter termed ‘growth recipients’, they can take any form or shape and can be open, closed or partly closed to the air and they can be open, closed or partly open to natural or man-made waterways. They can be either deep or shallow, depending on the circumstances and requirements. One simple example of their shape would be runways, comparable to what is commonly used in High Rate Algal Pond systems.

In one situation the water is pumped directly from the effluent channel which comes from the previous secondary treatment phase, and into the additional circulatory system for the ternary treatment, by means of standard commercially available centrifugal or peristaltic pumps. This ternary system may be attached or independent from the former treatment phases from which it is transported. After the water has been pumped through the system it is discharged back either into the said effluent channel or directly into the waterways. By extension, it is also possible to re-circulate part or all of the treated waste-water into the entire or part of ternary waste-water treatment procedure. This could be done for a shorter or longer period of time. This could be done for the purpose of bridging a temporary gap, either regular or irregular, in the otherwise continuous flow of waste-water coming from the waste-water treatment facility before the invention.

The ternary treatment of the waste-water comprises employing the waste-water within the ternary circulatory system as growth medium for communities of attached micro-organisms. In one embodiment these could comprise of a predominantly algal biomass. The waste-water may be used with or without the extra addition of chemical compounds (e.g. trace-elements), or the waste-water may or may not be additionally modified or pretreated by other means (e.g. UV irradiation, micro-waves, ultra-filtration).

After circulation through the invention, the treated waste-water will have been deprived of a part of the nutrient load (nitrate, phosphate, silicate), and other compounds, as a result of the biomass growth. This will enable the owner of the water purification plant to meet the required environmental standards even better e.g. lower nutrient or toxins loading of the water.

A part of the microbial communities comprise of algae, which are photosynthesizing organisms and are to be considered vegetal. Growth of vegetal biomass is referred to as primary production. Primary production is the natural process through which the incident energy of the sun is converted into biomass by the process of photosynthesis. For this process basic building blocks, or nutrients, are required by the organisms, these are the nutrients which are present in growth medium, in this case waste-water.

The ternary treatment discussed here comprise of growing micro-organisms within a continuous water flow. The waste-water is the transport-medium which brings the necessary nutrients to the micro-organisms. By simply growing (i.e. multiplying) these micro-organisms take up the nutrients in the waste-water and thus deplete the concentrations of these nutrients in the waste-water. Through this process of primary production the water is stripped of excess nutrients. In addition to depleting the nutrients, the microbial communities can fix or breakdown potentially hazardous compounds, either intra-cellular or in the matrix of the microbial communities.

The process of primary production also releases substantial amounts of oxygen, augmenting the concentration of oxygen in the water column. This is beneficial for all aquatic organisms that depend on respiration for their survival (e.g. fish) and is an important parameter that is commonly measured to determine water quality (e.g. Chemical Oxygen Demand COD, Biological Oxygen Demand BOD).

As stated earlier, in accordance with the present invention, the continuous waste-water stream is run across and through the artificial substrata which are simulacra's of the natural substrata where the biomass of micro-organisms grows under natural conditions (i.e. as can be found in for instance the rivers and channels of the region). The artificial substrata may be placed directly in the growth recipients or can be attached to some sort of carrier which is then placed in the growth recipients.

The artificial substrata are placed in such a way that the waste-water stream must go through and/or very close near them. Consequently, the waste-water comes in very close contact with the microbial communities which grow on the artificial substrata. The key issue here is maximizing the interaction interface between the growth medium and the microbial communities. The artificial substrata are placed in the circulatory system in such a way that they cannot be dislodged by the water current. The artificial substrata can be made of PVC, poly-carbonate, glass or natural organic material (e.g. horse hair). Any other material suitable for micro-organisms may be used as well.

Ideally, the artificial substrata are fractal or fractal-like (or fractaloid) in shape and form. A simple two dimensional plane is two dimensional (n=2), but can be considered to be fractaloid in as such that it's fractal nature is zero (n=2,0). Artificial substrata considered here can have a basis of one-, two- or three-dimensions in nature with an additional fractaloid dimension which may be zero (1,0; 2,0; 3,0) or larger than zero and smaller than 1. The main purpose is to maximize the available surface for attachment of micro-organisms within a given spatial volume while at the same time optimizing the flow through of the stream.

Ideally, the artificial substrata would be fully transparent, allowing for the photo-synthetically active radiation (PAR) to reach all positions up, around or in the substrata where photosynthesizing organisms would thrive.

The artificial substrata can be placed, removed and exchanged from the growth recipients of the circulatory system by an extracting device. This extracting device can be operated either manually, semi-automatically or fully automatically. The artificial substrata can be removed by either taking hold of them directly or by taking hold of the carrier to which they are attached. In one possible form the growth recipients have ridges along which a transportation device with a gripping, hooking or other attachment device may move in order to collect, place or exchange the artificial substrata from the recipients. In another form the retrieval device moves independently from and across the growth recipients and can either suspended from superstructure or move directly on the floor (e.g. a wheeled structure).

The microbial biomass grown on the artificial substrata is predominantly made up of micro-organisms that naturally occur in the aquatic systems of the region. These could for instance comprise of algae from the natural freshwater running or standing waters from the region. In accordance with the present invention, the directed growth of the microbial communities in this invention results in communities of micro-organisms that grow attached on the artificial substrata. These attached microbial communities can be further fine-tuned by governing various essential variables leading to continuous modular production of micro organism. If for instance it would be desired to create a dominance of a certain group of attached algae (e.g. Xanthophyta) in the microbial communities then this could be achieved by changing the settings of a number of essential variables such as, but not limited to, temperature, water velocity and light periodicity. This could be done for various purposes such as, but not limited to, changing community composition, diversity, productivity or chemical composition. Another example could be the directed removal of certain groups of organisms that are detrimental to the directed culturing of microbial communities of a desired composition. For instance, adjusting certain variables can result in adverse conditions for certain groups of grazers which consume the desired micro-organisms. Consequently, and as a result of the principle of CAS, the microbial community will exhibit diminished populations of these grazers.

Upon extracting the artificial substrata upon which the microbial communities have grown, they are transported to a harvesting device. In this device the microbial communities are separated from the artificial substrata. One example is that this could be done by using water jets. After separation the artificial substrata can then be recycled, with or without additional pretreatment before re-use. The separated microbial communities are primarily concentrated in the harvesting device by settling (i.e. gravitational forces) to the bottom where, if so desired, a collection recipient could be provided. The residual washing water, or super-natans, can be discarded by decanting, draining or any other means. The harvested microbial communities are taken from the harvesting machine and can then be further processed according to the desired end-product.

It is inherent to this type of crop in its original untreated form that it contains animal, vegetal and microbial organisms and can therefore not be called equivocally animal or vegetal. A first end-product generated by this invention is the process of water-purification.

The harvested microbial biomass is further processed depending on the desired additional end-products. It could either be sold as dry product in powder, tablet or other form (comparable to present ‘Spirulina’ market products) or in liquid form. Furthermore commercially interesting products may be extracted from the biomass which can then be marketed.

For instance, the algae present in the microbial biomass are rich in poly-unsaturated fatty acids (PUFA) of the omega 3 type (e.g. EPA, DHA). These products will be extracted and sold as food supplement. The algae may be also rich in pigments, such as astaxanthine, which could also be extracted and sold as food (human, animal), food-supplement, food adititives, pharmaceuticals and cosmetics. The targeted markets are both human food supplements and animal fodder both aquatic and terrestrial. Oil extracted from micro-algae is to be considered vegetal and is therefore an acceptable source for omega 3 oil for vegetarian diets. Other commercially interesting products may also be extracted and sold.

In addition, the microbial biomass can be used as an important source of renewable energy. The microbial biomass can be converted through standard techniques into electricity or biogas. It could also be converted into liquid oil (or bio-oil) through for instance the process of pyrolysis or thermo-chemical conversion. Oil can also be extracted from the biomass. This type of oil is commonly known as PPO: pure plant oil or PsPO, Pseudo Plant Oil. We may refer to it as algal oil.

It can be used directly as a liquid fuel for combustion or lubrication, mainly as a surrogate to petroleum and its derived products. Or the algal oil can be treated and converted into bio-fuel including bio-diesel, bio-gas and bio-ethanol. For instance this could be done through the process of trans-esterification. Other applications of biomass can be envisaged such as biomass fertilizers or raw material feedstock for chemical processing end products alternative for petrochemical products.

### BASIC DRAWINGS NUMBERED LEGEND

Drawings of the engineering part of the invention.

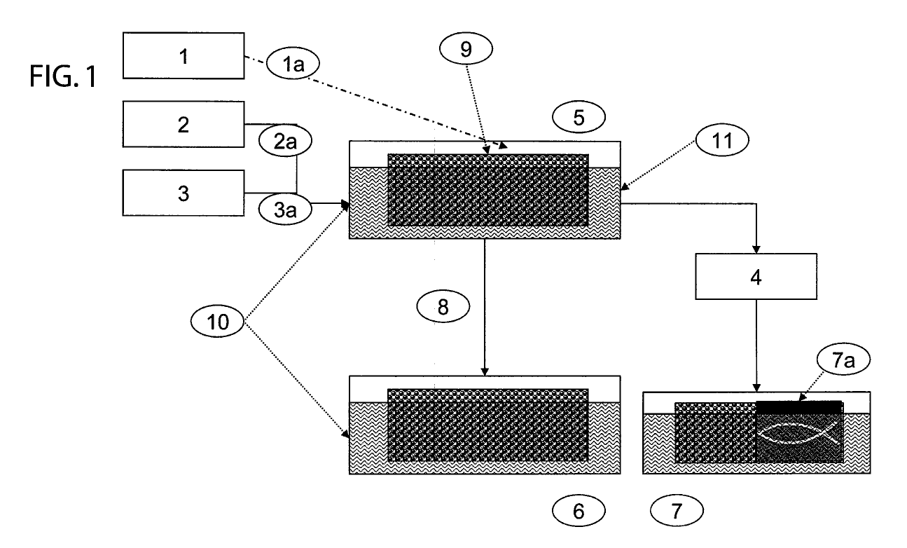

FIG. 1: Continuous flow of growth medium through growth recipient.
1: Light source of incident light, either artificial or natural.
1a: Incident light.
2: Influent water source which serves as growth medium for the micro-organisms e.g. waste-water.
2a: Influent water.
3: Source of nutrients added through various means (e.g. CO₂ as additional carbon source).
3a: Additional nutrients.
4: effluent water, discharged water after processing by invention.
5: T 0, time zero, temporal starting point of a de novo startup of the invention or of a newly inserted artificial substratum (9).
6: T 1, time one, first temporal point of harvesting the micro-organisms grown on the artificial substrata (9).
7: Any further application of the effluent water (4) e.g. (7a) as industrial water, growing fish, mollusca or any other living organism, . . .
8: The process of colonization and subsequent growth of the micro-organisms on the artificial substrata (9) between (5) and (6).
9: Artificial substrata. Artificial substrata are any substrate, wholly or partially submerged in the growth medium, on which the micro-organisms are attached and proliferate. The artificial substrata as such are detachable from the growth recipients, either as a whole or by means of a ‘carrier device’ on which the substrata are fixed (e.g. a metal frame).
10: Continuous water flow running over and across the artificial substrata.
11: The growth recipient. Any recipient through which the growth medium flows and in which the micro-organisms grow.

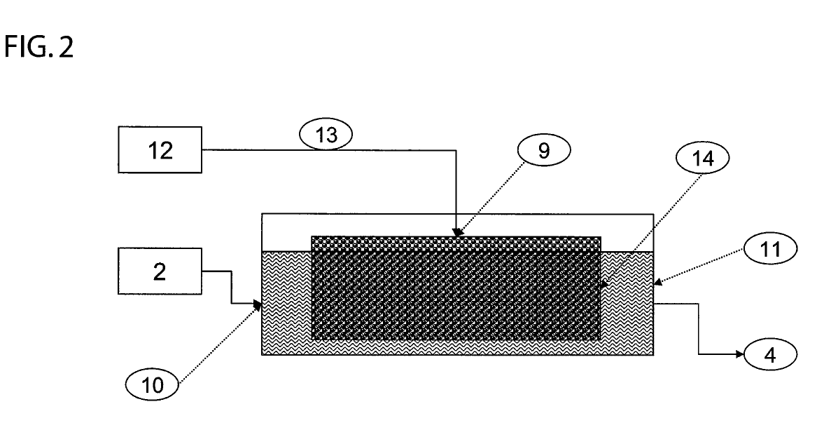

FIG. 2: Inoculation of artificial substrata by natural and diverse communities of micro-organisms.
12: Regional species pool. All micro-organisms within a given region that occur in the natural communities of that region and that may theoretically enter the growth recipient (both naturally or man-aided).
13: Continuous species rain. The continuous arrival in the growth recipient of species from the regional species pool.
14: The communities of micro-organisms that have successfully colonized the artificial substratum and that have been naturally selected by their environment. These communities intrinsically hold the ability to further adapt their species composition to changing environmental conditions.

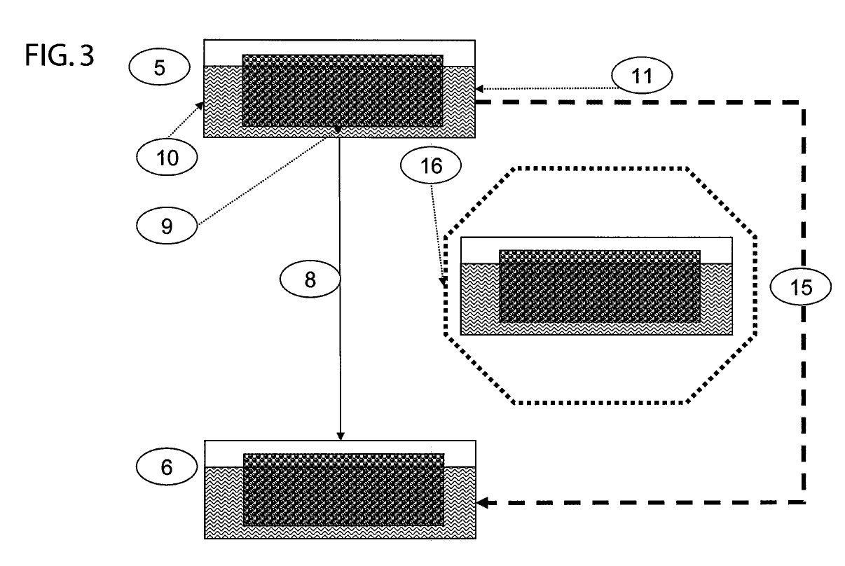

FIG. 3: Regulation of environmental variables in order to modulate the composition of the community of micro-organisms.
15: The same process as (8) while additionally governing various environmental variables in order to actively modulate the community composition, productivity and diversity. The resulting process consists of a controlled growth of the communities of micro-organisms.
16: All and any means or devices that are to be employed to accurately govern environmental variables within the growth recipients in correspondence with the requirements for (15).

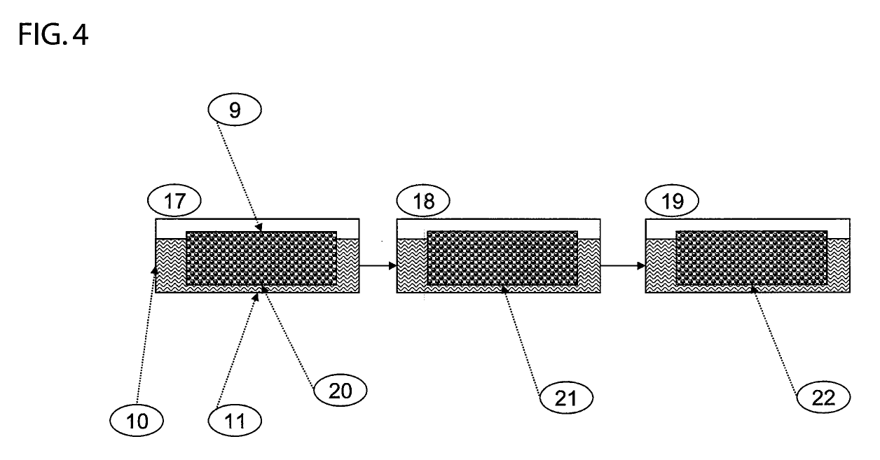

FIG. 4: Process for the growing of micro-organism in an open and continuous system according the present invention
17: P0 Spatial position zero. This is the theoretical first point of entry of the influent water that functions as growth medium.
18: P1 Spatial point 1. This is the theoretical first point in the growing recipient where a community of micro-organisms can successfully colonize the artificial substratum and develop a mature community. These spatial positions are here depicted as occurring discreetly in individual growth recipients but it is stressed that this could may also be found within one single large growth recipient containing several artificial substrata or even within one single artificial substratum.
19: PN Any given subsequent spatial point in the flow of the growth medium. Every point N is considered the spatial position where the community of micro-organisms substantially differs from the communities at point N−1 and point N+1 in regard of previously identified variables. These may be but are not limited to: species composition, diversity, dominance or concentration of chemical components. The physical distance between two spatial point defined as above may be different for each pair of positions.
20: Community at spatial point zero.
21: Community at spatial point 1.
22: Community at spatial point N.

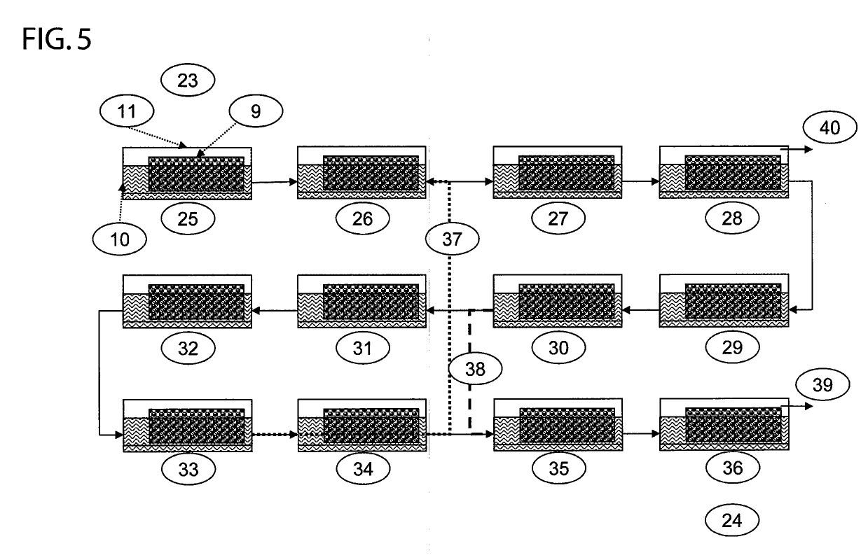

FIG. 5: Illustrating the process of intergrading communities and the application of feedbacks, shortcuts and fast exits in response to changing conditions and/or demands.
23: Position N. Any given position in the flow of water within the growth recipient, starting with spatial position 1 (17).
24: Position N+12. The drawing illustrates twelve discrete communities between spatial positions (23) and (24). The spatial position are depicted as occurring in discrete growth recipient, but is should be clear that these position might also occur within one growth recipient or even one artificial substratum.
25: Community of micro-organisms growing on the artificial substratum and at spatial position N with a given set of community properties such as, but not limited to, diversity, dominance, composition, concentration of chemical properties . . .
26: Community of micro-organisms at spatial position N+1 differing in at least one relevant variable from the communities at spatial position N and N+2.
27: Community of micro-organisms at spatial position N+2 differing in at least one relevant variable from the communities at spatial position N+1 and N+3.
28: Community of micro-organisms at spatial position N+3 differing in at least one relevant variable from the communities at spatial position N+2 and N+4.
29: Community of micro-organisms at spatial position N+4 differing in at least one relevant variable from the communities at spatial position N+3 and N+5.
30: Community of micro-organisms at spatial position N+5 differing in at least one relevant variable from the communities at spatial position N+4 and N+6.
31: Community of micro-organisms at spatial position N+6 differing in at least one relevant variable from the communities at spatial position N+5 and N+7.
32: Community of micro-organisms at spatial position N+7 differing in at least one relevant variable from the communities at spatial position N+6 and N+8.
33: Community of micro-organisms at spatial position N+8 differing in at least one relevant variable from the communities at spatial position N+7 and N+9.
34: Community of micro-organisms at spatial position N+9 differing in at least one relevant variable from the communities at spatial position N+8 and N+10.
35: Community of micro-organisms at spatial position N+10 differing in at least one relevant variable from the communities at spatial position N+9 and N+11.
36: Community of micro-organisms at spatial position N+11 differing in at least one relevant variable from the communities at spatial position N+9.
37: One possible example of feedback of growth medium from a later spatial position to an earlier spatial position. This feedback loop feeds growth medium, exiting from position N+9 (34) to community (26) on position N+1. This example shows a counter-current feedback, but it is understood that feedback may also occur following the normal flow of the growth medium.
38: One possible example of shortcutting the flow of growth medium from an earlier spatial position to a later non-adjoining spatial position. This shortcut loop feeds growth medium from spatial position N+5 (30) to community (35) at spatial position N+10.
39: The normal exit position where the growth medium is discharged as effluent.
40: One possible example of a fast exit which allows for the growth medium to be extracted or discharged from other positions than at the normal discharge exit of the growth medium as effluent (39).

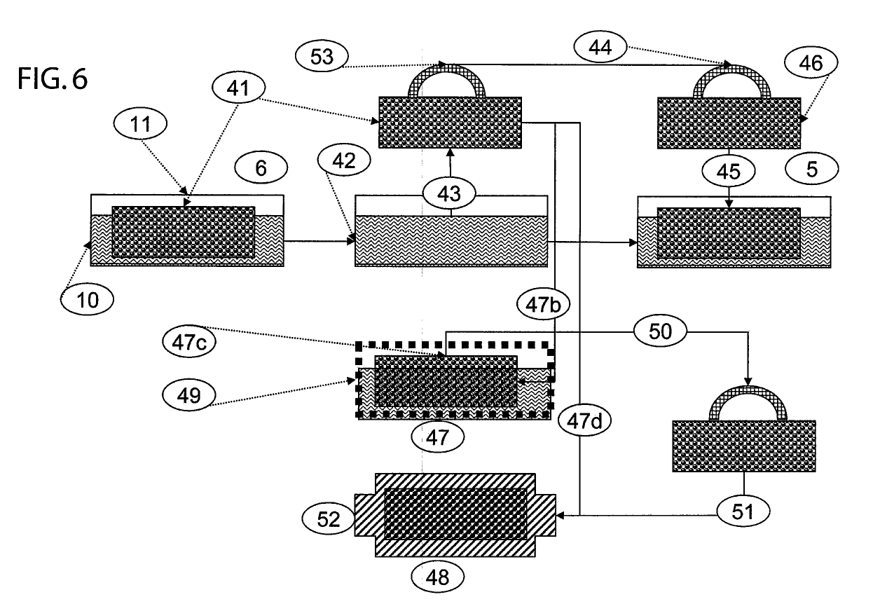

FIG. 6: Drawing illustrating the extraction of the artificial substrata.
41: Artificial substratum on which a community of micro-organisms has grown and has reached the stage of community development upon which it is to be harvested.
42: The continuous water flow does not have to be stopped during the extraction procedure.
53: Extracting device. The artificial substratum is extracted from the growth recipient by means of an extracting device.
43: The extraction device extracts the artificial substratum either by taking hold of the carrier upon which the artificial substratum is attached or by taking hold of the artificial substratum directly.
44: Replacing device. A replacing device brings a new artificial substratum. It is understood that this replacing device could be the same as the extracting device, but it could also be an individual device. Both devices may be features of a single machine, but not necessarily so.
45: The replacing device inserts the artificial substrata in the growth recipient without stopping the continuous water flow.
46: The new or recycled artificial substratum. This artificial substratum may be pretreated.
47: Two-step harvest procedure of micro-organisms. In order to maximize the production of certain compounds the micro-organisms it is possible to subject the biomass to an additional treatment before final harvesting.
47b: The carrier with the artificial substratum is transported to and placed in the treatment recipient (49). This transportation needs to occur timely, i.e. before any detrimental processes occur as a result of extracting the biomass from its growth medium.
47c: The carrier is submerged in another liquid medium which may or may not differ from the growth medium from which it was extracted (e.g. chemical composition). The treatment medium is not necessarily a flowing liquid or an open system.
47d: Alternatively a one-step harvest procedure may be followed wherein the biomass is directly processed in the harvesting machine and not pre-treated.
48: Actual harvesting process of the micro-organisms. The communities of micro-organisms are separated from the artificial substratum. This may happen in one single step removing the entire community or in separate steps each of which dealing with the removal of a specific part of the community (i.e. Certain groups) or of the biomass (i.e. Certain chemical compounds). These separate steps may encompass various biological, physical or chemical separation techniques.
49: Treatment recipient. In this recipient, that could be similar or different from a growth recipient, the biomass is treated for a shorter or longer period, typically hours to days.
50: After the desired treatment, the carrier with the biomass still attached to the artificial substratum is extracted by the extraction device (42).
51: The carrier with the artificial substratum is transported to and placed in the harvesting device (52). This transportation needs to occur timely, i.e. before any detrimental processes occur as a result of extracting the biomass from its growth medium.
52: The harvesting device separates the biomass of micro-organisms from the artificial substratum, see also (48).

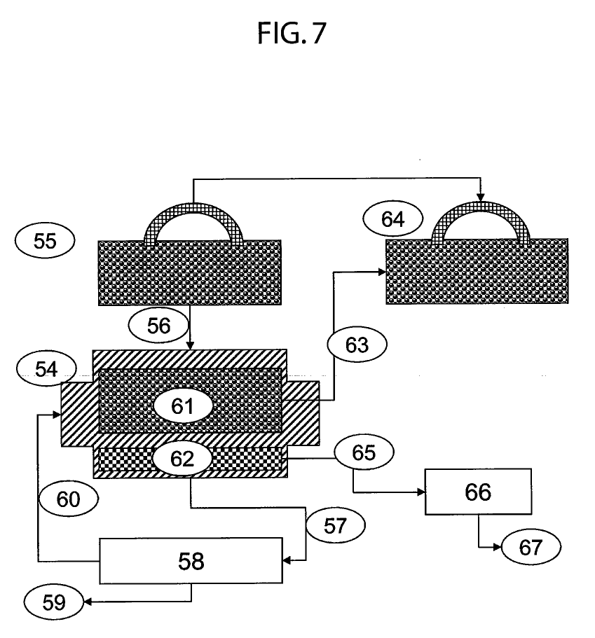

FIG. 7: Detailed scheme of separation of microbial communities from artificial substrata
54: Harvesting device or machine.
55: An artificial carrier grown with microbial communities is brought to the harvesting device by means of the extraction device.
56: The artificial carrier is inserted into the harvesting device.
57: After separation of the biomass from the artificial substrata the harvesting liquid (if so used) is discharged.
58: The harvesting liquid, if so used, is collected.
59: The harvesting liquid can be discharged
60: The harvesting liquid can be recycled, for instance in the harvesting device.
61: The biomass of microbial communities is separated from the artificial substrata
62: The biomass settles to the bottom through gravitation.
63: After separation the artificial carrier is extracted from the harvesting machine. This can be done by the extraction device.
64: The artificial carrier is either rejected or returned to the growth recipients.
65: The separated biomass is extracted from the harvesting machine after settling.
66: Biomass after separation.
67: The biomass is further processed.

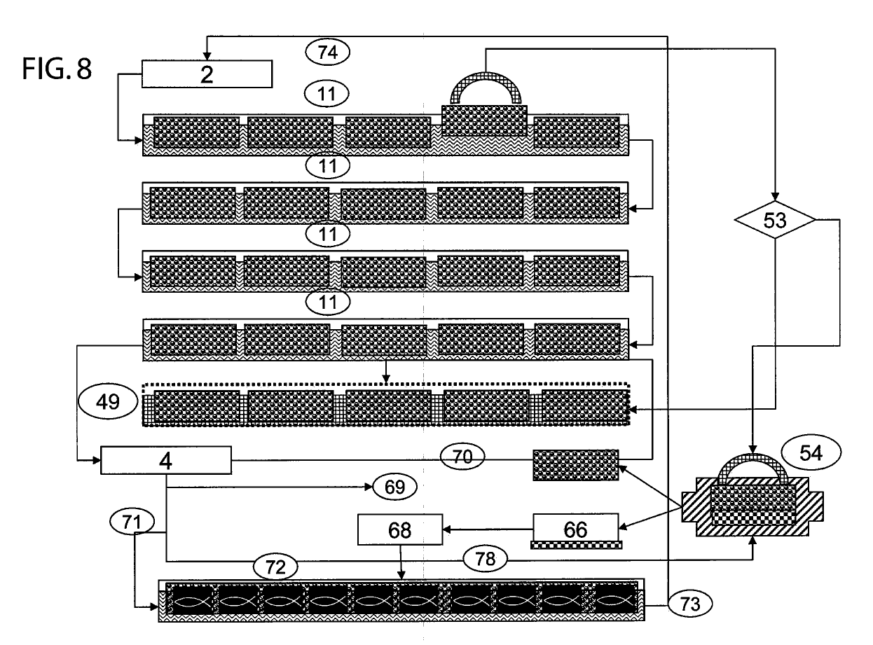

FIG. 8: One possible embodiment of the invention. Purifying waste-water from pisciculture facilities and recycling both the purified water and the grown biomass in the pisciculture.
68: Biomass processed into fish-fodder
69: Discharging effluent.
70: Using effluent as secondary treatment fluid.
71: Using effluent to grow fish in pisci-culture facility
78: Using effluent as harvesting liquid.
72: Pisci-culture facility.
73: Waste-water is discharged from pisci-culture facility.
74: Pisci-culture waste-water is collected in used as influent for the invention.

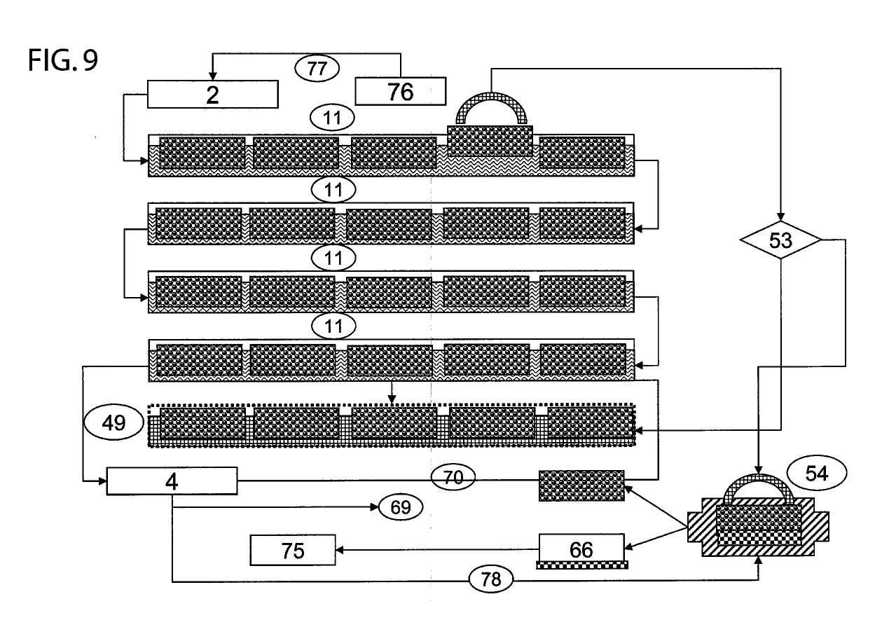

FIG. 9: One possible embodiment of the invention. Ternary treatment by microbial communities of the effluent from waste-water treatment facilities for domestic sewage.
75: Biomass processed into various end-products.
76: Waste-water treatment facility for domestic sewage.
77: Effluent of 76 is collected and used as influent for the invention.

### Drawings of a Prototype of One Possible Embodiment

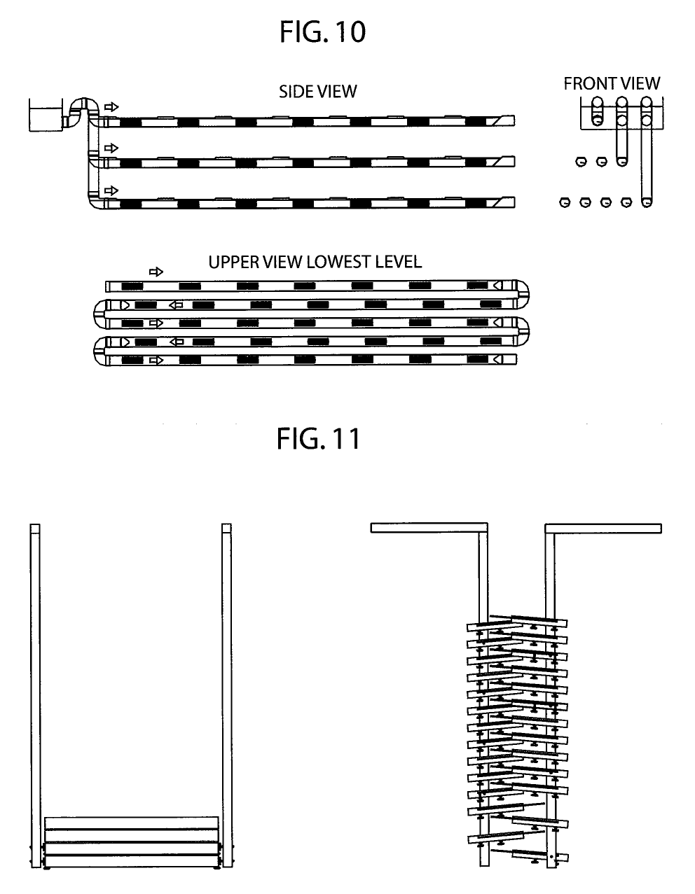

FIG. 10

Three layered prototype with pipelines through which the waste-water can flow. The artificial substrata are positioned within the pipes.

FIG. 11

Multi-layered cascade structure through which the waste-water can flow from top to bottom. The layers are formed by the artificial substrata.

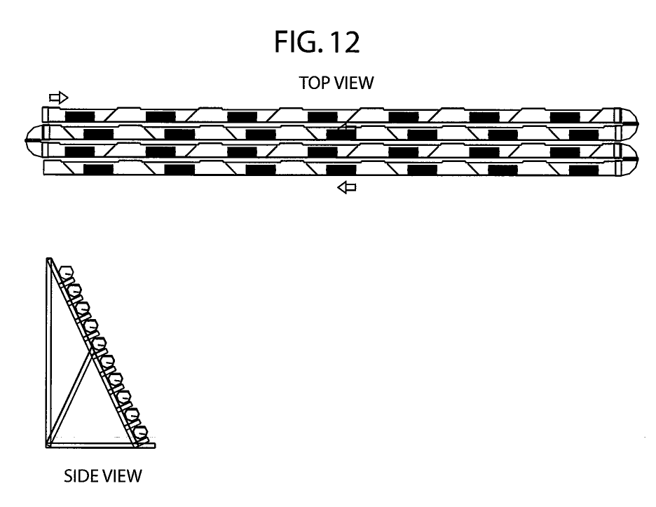

FIG. 12

Skewed and multilayered prototype with pipelines through which the waste-water can flow. The artificial substrata are positioned within the pipes.

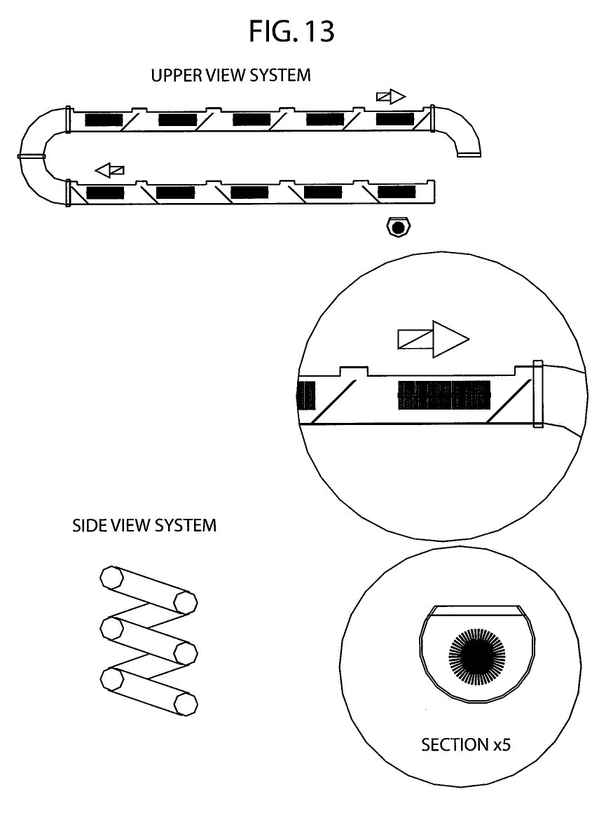

FIG. 13

Detail drawing of connecting pipeline system.

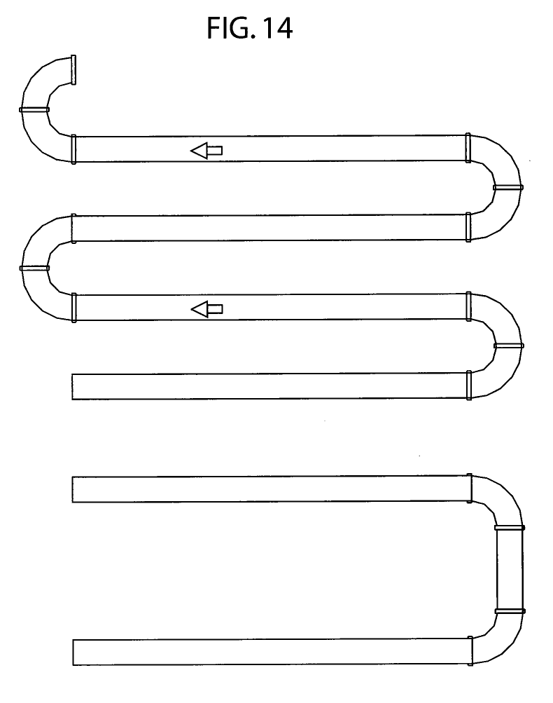

FIG. 14

Detail drawing of the positioning of the artificial substrata within the pipesystem.

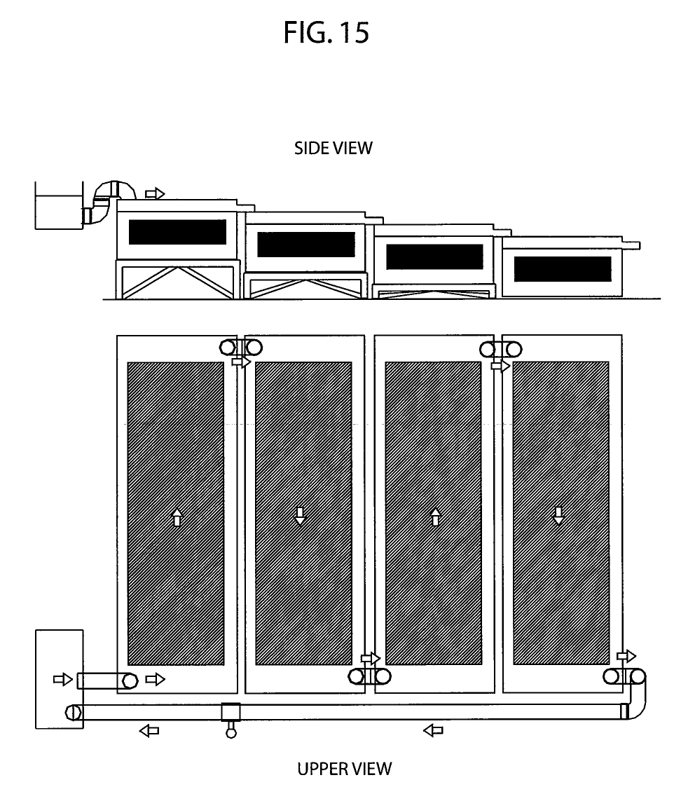

FIG. 15

Prototype with cascading growth recipients making use of gravity to move the waste-water stream. The artificial substrata are positioned in the flow of waste-water within the broad growth recipients.

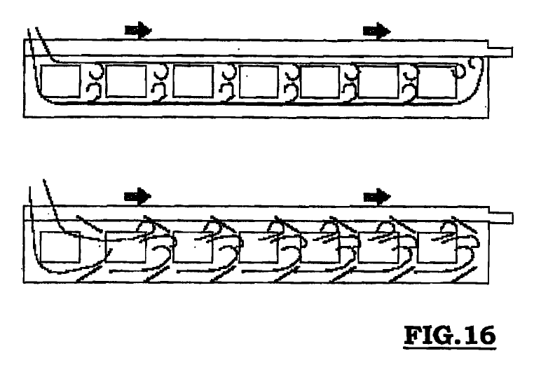

FIG. 16

This drawing schematically depicts the important role of the main stream of waste-water, the currents and under-currents and turbulence in guiding the water through or close-by the artificial substrata in the growth recipient.

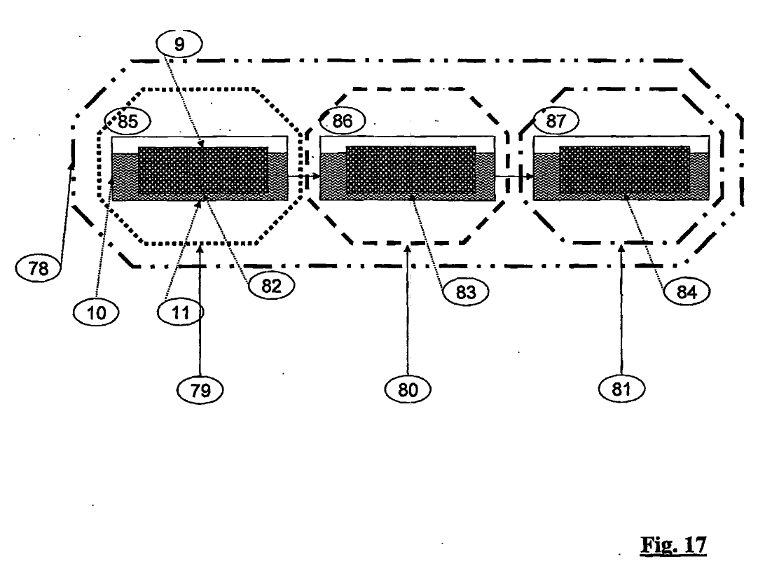

FIG. 17

An example of one embodiment illustrating the modularity and the application of the CAS mechanisms. Each system itself may comprise of one or more of a subsystem for example in parallel configuration.
78: General environmental configuration allowing for CAS to self-organise into a generally targeted community
79: Specific environmental subconfiguration A allowing for CAS to self-organise into a more specifically targeted community, with a particular productivity in view.
80: Specific environmental subconfiguration B allowing for CAS to self-organise into a more specifically targeted community, with a particular productivity in view.
81: Specific environmental subconfiguration C allowing for CAS to self-organise into a more specifically targeted community, with a particular productivity in view.
82: Resulting specific community A with targeted properties
83: Resulting specific community B with targeted properties
84: Resulting specific community C with targeted properties
85: Position A, at which local interactions are operational resulting in self-organisation.
86: Position B, at which local interactions are operational resulting in self-organisation.
87: Position C, at which local interactions are operational resulting in self-organisation.

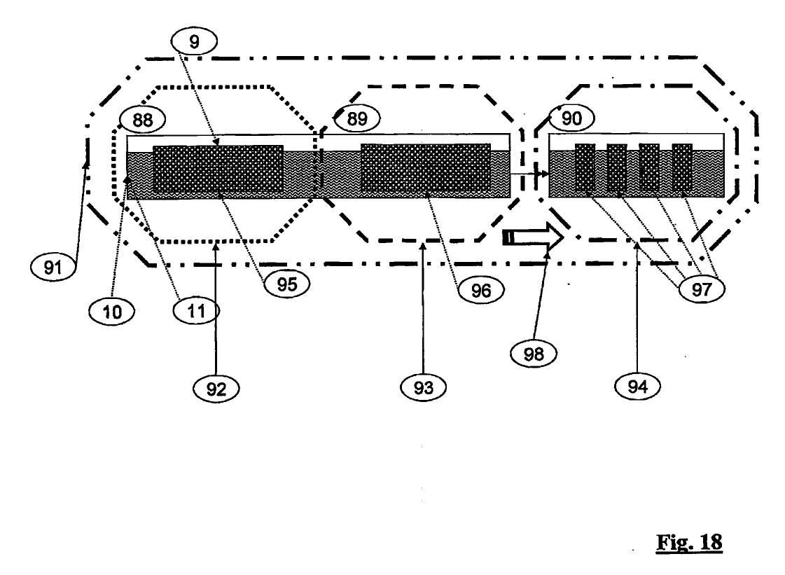

FIG. 18

An example of one embodiment illustrating the modularity and the application of the CAS mechanisms. This embodiment differs from to above in that a number of artificial substrata are placed in one system, while a next set of different artificial substrata are positioned in a next system, positioned downstream of the former.
88: Position A at which local interactions are operational resulting in self-organisation.
89: Position B at which local interactions are operational resulting in self-organisation.

Positions A and B are situated within one single system. The consist of two sets of artificial substrata within one single system.
90: Position C, consists of a series of adjacently positioned artificial substrata in one system.

At position C local interactions are operational resulting in self-organisation.
91: General environmental configuration allowing for CAS to self-organise into a generally targeted community
92: Specific environmental subconfiguration A allowing for CAS to self-organise into a more specifically targeted community, with a particular productivity in view.
93: Specific environmental subconfiguration B allowing for CAS to self-organise into a more specifically targeted community, with a particular productivity in view.
94: Specific environmental subconfiguration C allowing for CAS to self-organise into a more specifically targeted community, with a particular productivity in view.
95: Resulting specific community A with targeted properties
96: Resulting specific community B with targeted properties
97: Resulting specific community C with targeted properties. This community is distributed across a series of substrata, upon which growing sub-communities interact mutually.
98: Arrow symbolises the interaction between upstream communities A and B and downstream community C. Properties of Communities A and B affect Community C which according to CAS has adapted and consequently exhibits specifically targeted properties.

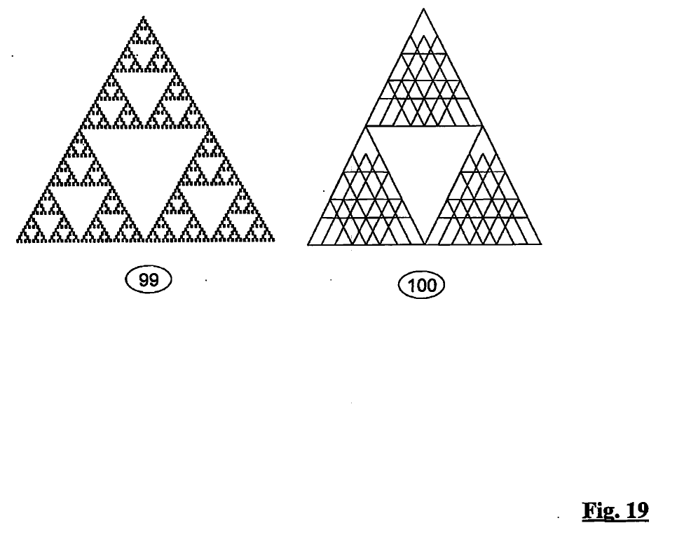

FIG. 19: Fractal pattern of the Serpienski Gasket (also translated Sierpinski) and simplified pattern of the Serpienski Gasket
99: Fractal pattern of the Serpienski Gasket (also translated Sierpinski)

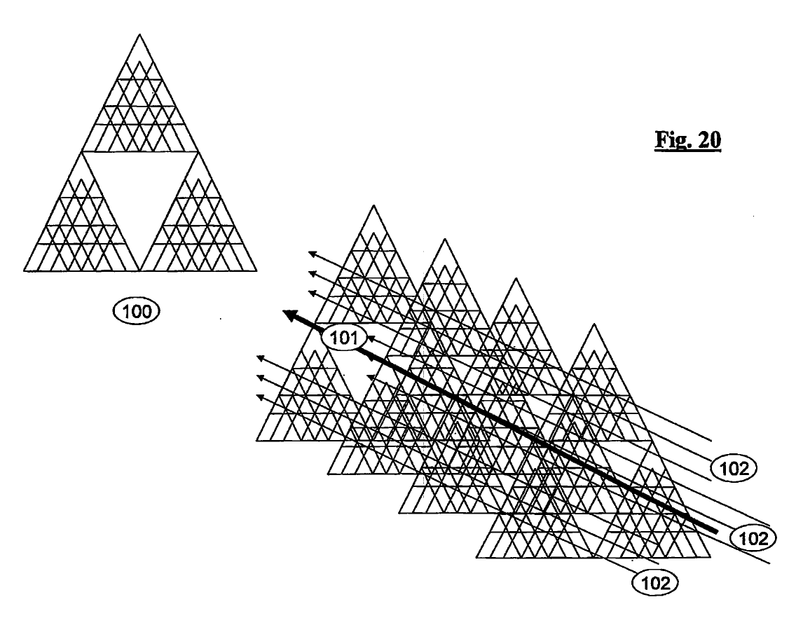

FIG. 20
100: Simplified pattern of the Serpienski Gasket
101: Symbolistation of the largest flow going through the artificial substrata
102/ Symbolistation of the smaller flows going through the artificial substrata, through the smaller openings of the substrata.

FIG. 21

Example of controlled algal growth of wild polycultures modulated through selected essential environmental parameters within a system. These communities comprise of a preferred group of organisms (e.g. Bacillariophyta) species composition (e.g. Fragilaria capucina)

1. Process for growing of micro-organisms in an open and continuous system which comprises creating within said system a habitat in which natural, diverse and heterogeneous microbial communities can autonomously react, self-organize and adapt to a changing environment

2. A process according to claim 1 which comprises providing at least one artificial substratum and growing microbial communities on said substratum.

3. A process according to claim 1 which comprises maintaining the fluidum stream continuous within the system.

4. A process according to claim 1 wherein said fluidum comprises a stream of waste water.

5. A process according to claim 2, further which comprises positioning the artificial substrata in the flow of the water stream and forcing the stream through or near the artificial substrata and the microbial communities attached to the substrata

6. A process according to claim 1, which comprises separate microbial communities present at different positions within the system.

7. A process according to claim 1 which comprises controlling one or more environmental variables to govern at least one property of the microbial communities at each and every position in the system.

8. A process for the production of micro-organisms according to claim 1 further comprising the step of harvesting the microbial communities by means of an extracting device to extract carriers holding the substrata.

9. A process for producing biomass comprising the process steps as defined in claim 1.

10. An extracting device for use in the production of micro-organism and/or biomass having means to transport the artificial substrata to a harvesting device comprising a carrier onto which the artificial substrata is mounted
